(19) United States
(12) Patent Application Publication
(10) Pub. No.: US 2009/0010894 A1
(43) Pub. Date: Jan. 8, 2009

(54) METHODS AND SYSTEMS FOR IDENTIFYING COMPOUNDS THAT MODULATE ALPHA-SYNUCLEIN AGGREGATION

(75) Inventor: J. William Langston, Los Altos Hills, CA (US)

Correspondence Address: WILSON SONSINI GOODRICH & ROSATI, 650 PAGE MILL ROAD, PALO ALTO, CA, 94304-1050, US

(73) Assignee: The parkinson's Institute

(21) Appl. No.: 12/147,402

(22) Filed: Jun. 26, 2008

Related U.S. Application Data
(63) Continuation-in-part of application No. 11/644,616, filed on Dec. 22, 2006.
(60) Provisional application No. 60/753,538, filed on Dec. 23, 2005.
(60) Provisional application No. 60/971,810, filed on Sep. 12, 2007.

(51) Int. Cl. A61K 35/76; A61K 38/02; A61K 31/7105; A61K 31/711; A61P 25/16; A61K 48/00; C40B 30/00

(52) U.S. Cl. 424/93.6; 514/2; 514/44; 506/7

(57) ABSTRACT

This invention relates to the field of determining the ability of agents to interfere with, reverse, or prevent α-synuclein aggregation and α-synuclein-related pathology and behavior. This invention also relates to methods for screening candidate agents for their potential as human medicaments.

### CROSS-REFERENCE

This application is a continuation-in-part of application Ser. No. 11/644,616, filed Dec. 22, 2006, which claims the benefit of U.S. Provisional Application No. 60/753,538 filed Dec. 23, 2005, both of which are incorporated herein by reference in their entirety and to which applications priority is claimed under 35 USC § 120. This application also claims the benefit of U.S. Provisional Application No. 60/971,810, filed Sep. 12, 2007, which application is also incorporated herein by reference in its entirety.

### TECHNICAL FIELD

Methods for identifying compounds that modulate synuclein aggregation are provided. Also provided are systems that include two or more methods for identifying compounds that modulate α-synuclein aggregation.

### BACKGROUND

α-synuclein is one of hundreds of proteins in mammalian cells, with expression in the brain and throughout the body. α-synuclein when misfolded creates fibrils. Fibrils can aggregate and are either too dense or too large to be flushed from the cell by lysosomes and/or proteasomes. As the α-synuclein protein aggregates, it can collect additional proteins and eventually turn into a “Lewy body” or a “Lewy neurite.” α-synuclein-related pathology is involved in the etiology of a variety of neurological disorders, including Parkinson's Disease, Parkinson's Disease with accompanying dementia, Lewy body dementia, Lewy body variant of Alzheimer's disease, Huntington's disease, Alzheimer's disease with Parkinsonism, and multiple system atrophy. Abnormal protein aggregates are a common pathological feature of many neurodegenerative diseases, as represented in Table 1.

Aggregation of α-synuclein may be due to overexpression and accumulation of the protein. Alternatively or concurrently, the aberrant shape or conformation of some molecules of α-synuclein may be impressed upon other synuclein molecules. These molecules then bind to one another and the protein aggregates accumulate and deposit inside the neuron, where they exert damage as they increase in size. This process first prevents the neuron from performing its necessary role in brain function and as it progresses, eventually kills the neuron.

Parkinson's disease has a prevalence of about 2% after age 65, and, thus, is one of the most common neurodegenerative human disorders. Its pathological hallmarks are: (a) the presence of Lewy bodies (Spillantini, et al., 1997; Nature 388:839-40), round cytoplasmic inclusions about 5-25 μm in diameter, mainly reactive for α-synuclein but also for ubiquitin and other proteins; and (b) massive loss of dopaminergic neurons in the pars compacta of the substantia nigra (Fearnley, et al., 1991; Brain 114:2283).

Effective treatments for neurodegenerative diseases such as Parkinson's disease are needed. Accordingly, a need exists for rapidly screening active agents to identify those that specifically prevent, inhibit or disperse the pathological aggregation of α-synuclein.

### SUMMARY

Provided herein are methods and systems for identifying agents that modulate α-synuclein aggregation. The methods of the present invention are useful to determine the anti-aggregation potential of agents or to screen for agents with anti-aggregation or dis-aggregating properties.

In one embodiment, a method for identifying an agent that modulates α-synuclein aggregation is provided. The methods included providing a plurality of candidate agents and 1) contacting at least one candidate agent with a polypeptide comprising α-synuclein and 2) determining whether the agent promotes α-synuclein disaggregation, or inhibits α-synuclein aggregation, in vitro. The method further includes administering an agent identified in the in vitro assay to a non-mammalian organism comprising neuronal cells that overexpress a polypeptide comprising α-synuclein and determining whether the agent promotes α-synuclein disaggregation, or inhibits α-synuclein aggregation, by-detecting a change in the aggregation of the overexpressed α-synuclein. The method also includes administering an agent identified in the non-mammalian in vivo assay to a transgenic mammalian organism comprising neuronal cells that overexpress a polypeptide comprising α-synuclein in vivo and determining whether the agent promotes α-synuclein disaggregation, or inhibits α-synuclein aggregation, by detecting a change in the aggregation of the overexpressed α-synuclein. The method optionally includes administering an agent identified in the transgenic non-human mammalian assay to a non-human primate genetically modified to overexpress a polypeptide comprising α-synuclein in vivo, or treated with a chemical to induce Parkinson's disease-like state, and determining whether the agent promotes α-synuclein disaggregation, or inhibits α-synuclein aggregation by detecting a change in the aggregation of the overexpressed α-synuclein.

In another embodiment, a method for identifying an agent that modulates α-synuclein aggregation is provided. The methods included provide a plurality of candidate agents and 1) contacting at least one candidate agent with a polypeptide comprising α-synuclein and 2) determining whether the agent promotes α-synuclein disaggregation, or inhibits α-synuclein aggregation, in vitro. The method also includes administering an agent identified in the in vitro assay to a transgenic mammalian organism comprising neuronal cells that overexpress a polypeptide comprising α-synuclein in vivo and determining whether the agent promotes α-synuclein disaggregation, or inhibits α-synuclein aggregation, by detecting a change in the aggregation of the overexpressed α-synuclein. The method optionally includes administering an agent identified in the transgenic non-human mammalian assay to a non-human primate genetically modified to overexpress a polypeptide comprising α-synuclein in vivo, or treated with a chemical to induce Parkinson's disease-like state, and determining whether the agent promotes α-synuclein disaggregation, or inhibits α-synuclein aggregation by detecting a change in the aggregation of the overexpressed α-synuclein.

In one embodiment, a method for identifying an agent that modulates α-synuclein aggregation is provided. The methods included provide a plurality of candidate agents and 1) contacting at least one candidate agent with a polypeptide comprising α-synuclein and 2) determining whether the agent promotes α-synuclein disaggregation, or inhibits α-synuclein aggregation, in vitro. The method further includes administering an agent identified in the in vitro assay to a non-mammalian organism comprising neuronal cells that overexpress a polypeptide comprising α-synuclein and determining whether the agent promotes α-synuclein disaggregation, or inhibits α-synuclein aggregation, by-detecting a change in the aggregation of the overexpressed α-synuclein. The method optionally includes administering an agent identified in the transgenic non-mammalian assay to a non-human primate genetically modified to overexpress a polypeptide comprising α-synuclein in vivo, or treated with a chemical to induce Parkinson's disease-like state, and determining whether the agent promotes α-synuclein disaggregation, or inhibits α-synuclein aggregation by detecting a change in the aggregation of the overexpressed α-synuclein.

In one embodiment, a method for identifying an agent that modulates α-synuclein aggregation is provided. The method includes administering at least one candidate agent to a non-mammalian organism comprising neuronal cells that overexpress a polypeptide comprising α-synuclein and determining whether the agent promotes α-synuclein disaggregation, or inhibits α-synuclein aggregation, by-detecting a change in the aggregation of the overexpressed α-synuclein. The method also includes administering an agent identified in the non-mammalian in vivo assay to a transgenic mammalian organism comprising neuronal cells that overexpress a polypeptide comprising α-synuclein in vivo and determining whether the agent promotes α-synuclein disaggregation, or inhibits α-synuclein aggregation, by detecting a change in the aggregation of the overexpressed α-synuclein. The method optionally includes administering an agent identified in the transgenic non-human mammalian assay to a non-human primate genetically modified to overexpress a polypeptide comprising α-synuclein in vivo, or treated with a chemical to induce Parkinson's disease-like state, and determining whether the agent promotes α-synuclein disaggregating, or inhibits α-synuclein aggregation by detecting a change in the aggregation of the overexpressed α-synuclein.

In one embodiment of the invention, the neuronal cells are dopaminergic cells. In some aspects, the neuronal cells are genetically modified to overexpress α-synuclein. In some embodiments, the α-synuclein is human α-synuclein. In other embodiments, the overexpressed α-synuclein is mutant α-synuclein. In some aspects, the mutant α-synuclein is mutant human α-synuclein A53T. In other aspects the mutant α-synuclein is mutant human α-synuclein A30P. In still other aspects, the mutant α-synuclein is mutant human α-synuclein E46K.

In another embodiment, the organism used in methods provided herein is Caenorhabditis elegans (C. elegans). In other embodiments, the organism is yeast, bacteria or another microorganism. In still other embodiments, the organism is a member of the genus Drosophila. In still other embodiments, the organism is a mouse or a rat.

In one embodiment, mammalian or non-mammalian organisms are transgenic. In other embodiments, organisms are treated to induce Parkinson's-like disease with MPTP.

In some aspects of the invention, the detection of α-synuclein aggregation is by microscopy. In other aspects, the detection is by immunofluorescence.

In one embodiment, agents identified by methods of the invention are suitable for treating a condition associated with α-synuclein aggregation.

In other embodiments, the agent is selected from the group consisting of a chemical, a therapeutic molecule, a biomolecule, and a virus. The chemical may be a small molecule. The therapeutic molecule may be any therapeutic molecule, such as an antibiotic. Moreover, the biomolecule may be a polypeptide. In some aspects, the polypeptide is a peptoid. In other aspects the biomolecule is a nucleic acid such as DNA or RNA. In some aspects, the RNA may be anti-sense RNA. In other aspects, the RNA is siRNA.

In another embodiment, a method of treating an individual suffering from a disease associated with α-synuclein aggregation is provided. The method includes administering to the individual a pharmaceutical composition comprising a therapeutically effective amount of an agent identified by a method or system provided herein.

In another embodiment, a method of treating an individual suffering from Parkinson's disease is provided. The method includes administering to the individual a pharmaceutical composition comprising a therapeutically effective amount of an agent identified by a method or system provided herein.

In another embodiment, a system for identifying an agent that modulates α-synuclein aggregation is provided. The system includes practicing a method provided herein and communicating the results of the method to a database. The results may be accessed by multiple users. In some aspects, the information is optionally correlated with information contained in other databases.

In another embodiment a method for identifying an agent is provided. The steps of the method include: contacting a plurality of candidate agents with a polypeptide comprising α-synuclein and identifying agents that promote α-synuclein disaggregation, or inhibit α-synuclein aggregation upon contact with said agent, in vitro; administering one or more agents identified in vitro to a non-mammalian organism exhibiting damaged dopaminergic neuronal cells which comprise α-synuclein and identifying agents that promote α-synuclein disaggregation, or inhibit α-synuclein aggregation; and administering at least one agent identified in the non-mammalian organism to a mammalian organism comprising neuronal cells that overexpress a polypeptide comprising α-synuclein and determining whether the agent promotes α-synuclein disaggregation, or inhibits α-synuclein aggregation.

In another embodiment a method for identifying an agent includes contacting a plurality of candidate agents with a polypeptide comprising α-synuclein and identifying agents that promote α-synuclein disaggregation, or inhibit α-synuclein aggregation upon contact with said agent, in vitro; administering one or more agents identified in vitro to a non-mammalian organism comprising neuronal cells that overexpress a polypeptide comprising α-synuclein and identifying agents that promote α-synuclein disaggregation, or inhibit α-synuclein aggregation; and administering at least one agent identified in a non-mammalian organism to a mammalian organism exhibiting damaged dopaminergic neuronal cells which comprise α-synuclein and determining whether the agent promotes α-synuclein disaggregation, or inhibits α-synuclein aggregation.

In yet another embodiment a method for identifying an agent is provided, the steps of the method include: contacting a plurality of candidate agents with a polypeptide comprising α-synuclein and identifying agents that promote α-synuclein disaggregation, or inhibit α-synuclein aggregation upon contact with said agent, in vitro; administering one or more agents identified in the in vitro assay to a non-mammalian organism exhibiting damaged dopaminergic neuronal cells which comprise α-synuclein and identifying agents that promote α-synuclein disaggregation, or inhibit α-synuclein aggregation; administering at least one agent identified in the non-mammalian organism to a mammalian organism exhibiting damaged dopaminergic neuronal cells which comprise α-synuclein and determining whether the agent promotes α-synuclein disaggregation, or inhibits α-synuclein aggregation.

In yet another embodiment a method for identifying an agent is provided, the steps include: contacting a plurality of candidate agents with a polypeptide comprising α-synuclein and identifying agents that promote α-synuclein disaggregation, or inhibit α-synuclein aggregation upon contact with said agent, in vitro; and administering one or more agents identified in vitro to a non-mammalian organism exhibiting damaged dopaminergic neuronal cells which comprise α-synuclein and determining whether the agent promotes α-synuclein disaggregation, or inhibits α-synuclein aggregation.

In yet another embodiment a method for identifying an agent is provided, where the method includes: contacting a plurality of candidate agents with a polypeptide comprising α-synuclein and identifying agents that promote α-synuclein disaggregation, or inhibit α-synuclein aggregation upon contact with said agent, in vitro; and administering at least one agent identified in vitro to a mammalian organism exhibiting damaged dopaminergic neuronal cells which comprise Oα-synuclein and determining whether the agent promotes α-synuclein disaggregation, or inhibits α-synuclein aggregation.

In yet another embodiment, a method for identifying an agent is provided, and includes: administering a plurality of agents to a non-mammalian organism exhibiting damaged dopaminergic neuronal cells which comprise α-synuclein and identifying agents that promote α-synuclein disaggregation, or inhibit α-synuclein aggregation; and administering at least one agent identified in the non-mammalian organism to a mammalian organism comprising neuronal cells that overexpress a polypeptide comprising α-synuclein and determining whether the agent promotes α-synuclein disaggregation, or inhibits α-synuclein aggregation.

In yet another embodiment a method for identifying an agent is provided, including: administering a plurality of agents to a non-mammalian organism comprising neuronal cells that overexpress a polypeptide comprising α-synuclein and identifying agents that promote α-synuclein disaggregation, or inhibit α-synuclein aggregation; and administering at least one agent identified in the non-mammalian organism to a mammalian organism exhibiting damaged dopaminergic neuronal cells which comprise α-synuclein and determining whether the agent promotes α-synuclein disaggregation, or inhibits α-synuclein aggregation.

In yet another embodiment a method for identifying an agent is provided and includes: administering a plurality of agents to a non-mammalian organism exhibiting damaged dopaminergic neuronal cells which comprise α-synuclein and identifying agents that promote α-synuclein disaggregation, or inhibit α-synuclein aggregation; and administering at least one agent identified in the non-mammalian organism to a mammalian organism exhibiting damaged dopaminergic neuronal cells which comprise α-synuclein and determining whether the agent promotes α-synuclein disaggregation, or inhibits α-synuclein aggregation.

In some embodiments the method of treating an individual suffering from a disease associated with α-synuclein aggregation or Parkinson's disease is provided and includes administering to the individual a pharmaceutical composition comprising a therapeutically effective amount of an agent identified.

In some aspects, the embodiments described herein could be followed by further administering an identified agent to a non-human primate overexpressing a polypeptide comprising α-synuclein or to a non-human primate exhibiting damaged dopaminergic neuronal cells which comprise α-synuclein and determining whether the agent promotes α-synuclein disaggregation, or inhibits α-synuclein aggregation. The non-human primate can be a gorilla, baboon, chimpanzee, macaque, rhesus monkey, or a squirrel monkey.

In other aspects the identified compounds are communicated to a database. In yet another aspect of the embodiments described herein, the promotion of disaggregation, or the inhibition of aggregation, by the agent identifies the agent as a compound suitable for treating a condition associated with α-synuclein aggregation. In some aspects of the embodiments described herein, the agent is selected from the group consisting of a chemical, a small molecule, a biomolecule, and a virus.

In certain aspects, the overexpression of α-synuclein is achieved by AAV-mediated delivery of wild type α-synuclein, or mutant human α-synuclein carrying the A53T, E46K, or A30P mutations. In other aspects, the damage to dopaminergic neurons is result of administering a neurotoxin such as MPTP or 6-OHDA.

In the embodiments described herein, the mammalian organism is a rodent, a transgenic rodent, in some cases a transgenic rat or mouse. In other embodiments described herein, the non-mammalian organism is selected from the group consisting of worm (C. elegans), fly (D. melanogaster), fish (D. rerio), yeast, or bacteria.

In the embodiments described herein, determining whether the agent promotes α-synuclein disaggregation, or inhibits α-synuclein aggregation is by microscopy, measurement of mitochondrial activity or stress, measurement of oxidative stress, neuronal function, neuronal physiology, axonal transport, neurotransmission, neuronal transporter function or by detecting a change in behaviors such as: locomotor behavior, swimming behavior, touch response, and feeding behavior.

In the embodiments described herein when an in vitro system is utilized, it can be a primary neuronal culture or a neuronal cell lines such as SH-SY5Y cells. In certain embodiments, a kit is provided for the screening of a library of candidate agents. The kit can include purified α-synuclein and the components of a cell-free system. The kit could alternatively include purified α-synuclein or a plasmid for overexpression of α-synuclein and a library of candidate agents. The details of one or more embodiments of the disclosure are set forth in the accompanying drawings and the description below. Other features, objects, and advantages will be apparent from the description and drawings, and from the claims.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow diagram depicting one embodiment of the invention for screening agents that modulate α-synuclein aggregation or α-synuclein-related neuronal physiology or behavior.

FIG. 2 is a flow diagram depicting another embodiment of the invention for screening agents that modulate α-synuclein aggregation or α-synuclein related neuronal physiology or behavior.

### DETAILED DESCRIPTION

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The practice of the present invention employs, unless otherwise indicated, conventional techniques of neurobiology, neurochemistry, neurology, immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Described herein are methods and systems for identifying candidate therapeutic agents that modulate the aggregation of α-synuclein. Examples of therapeutic agents include any biologically, physiologically, or pharmacologically active substances that act locally or systemically in a subject. Therapeutic agents include for example, drugs such as those described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Therapeutic agents also include, for example, small molecules, steroids and esters of steroids, boron-containing compounds, chemotherapeutic nucleotides, antibiotics, antivirals, antifungals, enediynes, heavy metal complexes (e.g., cisplatin), hormone antagonists, non-specific (non-antibody) proteins, sugar oligomers, oligonucleotides (e.g., antisense oligonucleotides that bind to a target nucleic acid sequence (e.g., mRNA sequence)), siRNA, peptides, proteins, antibodies, photodynamic agents (e.g., rhodamine 123), radionuclides (e.g., 1-131, Re-186, Re-188, Y-90, Bi-212, At-211, Sr-89, Ho-166, Sm-153, Cu-67 and Cu-64), toxins (e.g., ricin), and transcription-based pharmaceuticals.

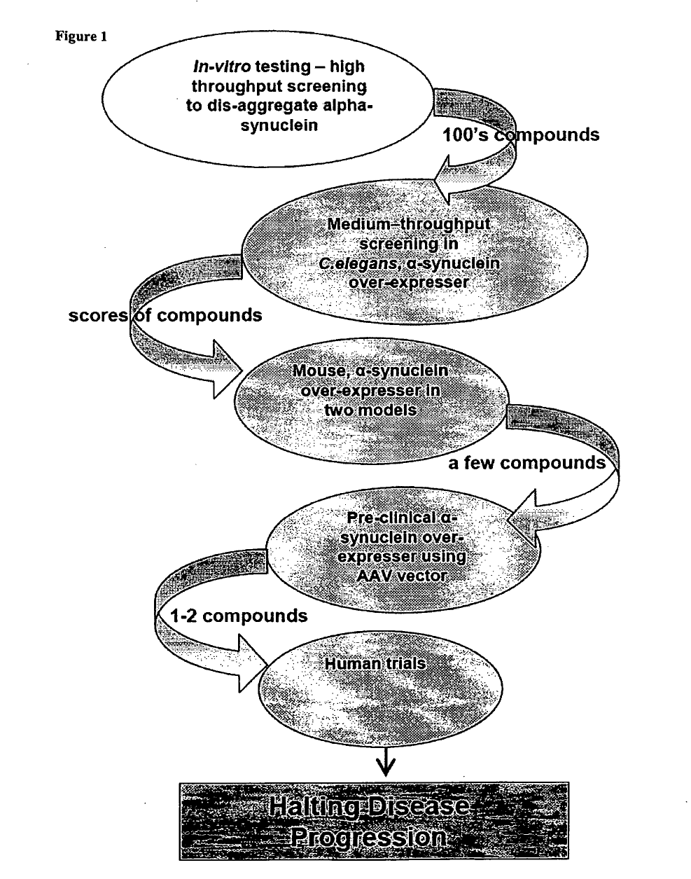

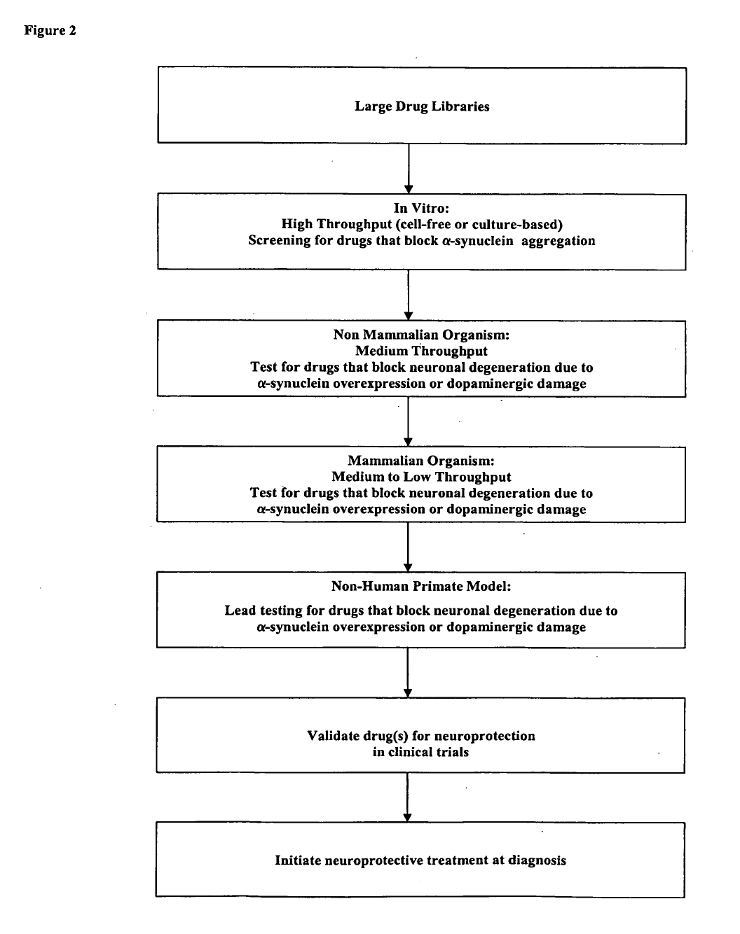

Referring to FIGS. 1 and 2, a library containing a plurality of candidate agents may be evaluated to determine the most desirable subsets, or “best-in-class” members. Such libraries can be generated on the basis of their expected binding affinities to α-synuclein, and derivatives thereof and modulating activities of α-synuclein and derivatives thereof. Desirable subsets (i.e., lead candidate agents) are then screened in subsequent assays to identify those that display optimal α-synuclein modulating activity. In this manner, successive rounds of screening can be used to identify the agent that effectively modulates α-synuclein activity.

“Modulation of α-synuclein aggregation” includes prevention, inhibition or decrease, change in time of onset, progress to or reversal of aggregation. Thus, “modulation” includes promoting the disaggregation of α-synuclein aggregates. The term “α-synuclein” includes mutant and wild-type polypeptides, and fragments thereof. An “activity” or “function” of α-synuclein includes, but is not limited to, formation of inclusions/aggregation in the cytoplasm, association with the cell membrane, interaction with an α-synuclein associated protein. In addition, α-synuclein can inhibit phospholipase D (PLD) activity, cause toxicity to cells, and lead to impaired proteasomal activity. For example, the identified agent may prevent α-synuclein misfolding, inhibit formation of α-synuclein inclusions/aggregation, or promote α-synuclein disaggregation. Accordingly, irrespective of the mechanism of action, agents identified by the screening methods described herein will provide therapeutic benefit to α-synuclein associated diseases.

FIGS. 1 and 2 provide a general description of methods and systems used to screen and identify agents that modulate α-synuclein aggregation. Initially, the ability of various agents to modulate α-synuclein aggregation in vitro can be tested. Since α-synuclein can be grown and aggregated in a cell culture, an exemplary assay may include contacting aggregated α-synuclein with a candidate agent and determining whether the agent promotes disaggregation of α-synuclein. Exemplary agents shown to have such an effect include rifampin, rifampicin, rifamycin, or rifaldazine. It is understood that α-synuclein aggregation may occur in the presence or absence of a cellular environment, a “cell-free” in vitro assay. See, e.g., Kim et al., (2006) J. Biol. Chem.: 281 (33250-57). For example, international application WO00/20020 (Masliah; published Apr. 13, 2000) describes methods of screening α-synuclein anti-aggregating compounds using metal-induced α-synuclein aggregation and Thioflavin-S staining. In vitro systems can include cell-free, cell-intact, cell culture, ex vivo, culture, cell culture, slice culture, primary cell line, or modified cell line.

Accordingly, certain aspects of the present disclosure provide methods and assays of screening for a candidate therapeutic agent (drug, compound or any of the other therapeutic agents named herein) and identifying an agent for treating a condition or disease associated with α-synuclein aggregation. In some embodiments, a subject can be treated prior to displaying any symptoms associated with α-synuclein aggregation. A “candidate agent” as used herein, is any substance with a potential to reduce, reverse, interfere with or block α-synuclein aggregation. Aggregation may be attributable to overexpression of α-synuclein or expression of abnormally processed or mutant α-synuclein. Various types of candidate agents may be screened by the methods described herein, including nucleic acids, polypeptides, small molecule compounds, and peptidomimetics.

Candidate agents include chemicals (including polymers, organic compounds, etc.); therapeutic molecules (including therapeutic drugs, antibiotics, etc.); biomolecules (including hormones, cytokines, proteins, lipids, carbohydrates, cellular membrane antigens); receptors (neural, hormonal, nutrient, and cell surface receptors) or their ligands; and viruses (including retroviruses, herpes viruses, adenoviruses, lentiviruses, etc.).

Candidate agents may be screened from large libraries of synthetic or natural compounds. One example is an FDA approved library of compounds that can be used by humans. In addition, synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.), and a rare chemical library is available from Aldrich (Milwaukee, Wis.). Combinatorial libraries are available and can be prepared. Alternatively, libraries of natural compounds in the form of bacterial, fungal, plant and animal extracts are also available, for example, Pan Laboratories (Bothell, Wash.) or MycoSearch (NC), or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. It should be understood, although not always explicitly stated that the agent can be used alone or in combination with another modulator, having the same or different biological activity as the agents identified by the subject screening method. Several commercial libraries can immediately be used in the screens.

Candidate agents may include a small molecule. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics.

In other embodiments, candidate agents also encompass numerous chemical classes, though typically they are organic molecules, often small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulphydryl or carboxyl group.

Other suitable candidate agents may include antisense molecules, ribozymes, and antibodies (including single chain antibodies), each of which would be specific for the target molecule. For example, an antisense molecule that binds to a translational or transcriptional start site, or splice junctions, would be ideal candidate inhibitors.

In one embodiment, initial screening methods described herein may include any assay that provides for the identification of agents that prevent α-synuclein misfolding, inhibit formation of α-synuclein inclusions/aggregation or promote α-synuclein disaggregation. Some of these assays may be cell-free in vitro assays. Detection of the level of α-synuclein aggregation in such systems may be measured by turbidity and or fluorescence detection (e.g., Thioflavin T). See, e.g., Sung et al., (2005) J. Biol. Chem.: 280 (25216-24). In other embodiments, electron microscopy will be used to visualize fibrillation and/or aggregation. Additionally centrifugation can be utilized in some embodiments to separate soluble and insoluble protein, followed by SDS-PAGE electrophoresis. In other embodiments atomic force microscopy images can be processed and viewed.

Other embodiments contemplate screening assays using fluorescence activated cell sorting (FACS) analysis. FACS is a technique well known in the art, and provides the means for scanning individual cells for the presence of fluorescently labeled/tagged moiety. The method is unique in its ability to provide a rapid, reliable, quantitative, and multiparameter analysis on either living or fixed cells. For example, α-synuclein can be suitably labeled using a variety of fluorescent tags (e.g., ThioflavinT, fluorescent antibodies, etc.) providing a useful tool for the analysis and quantitation of α-synuclein aggregation and fibril and/or aggregate formation.

In some embodiments, a cell free in vitro assay can be followed by or substituted with an in vitro primary culture assay. In this assay, primary neuronal cultures consisting of pure dopaminergic neurons, mixed primary cultures with dopaminergic neurons and glial cells made to express or overexpress wild-type or mutant α-synuclein or a neuronal cell line stably transfected with a wild-type or mutant α-synuclein can be utilized for screening.

In other embodiments, methods of the present disclosure relate to determining proteasomal impairment caused by α-synuclein. In yet other embodiments, methods of the present disclosure relate to determining oxidative stress caused by α-synuclein. Mitochondrial dysfunction and oxidative stress are clearly linked to diseases (e.g., Parkinson's disease) but in ways still poorly understood. Mitochondrial function, dysfunction and oxidative stress can be determined in a variety of methods well known in the art. These assays can be, but are not limited to an (3-(4,5-Dimethylthiazol-2-yl)-2,5-diphenyltetrazolium bromide, a tetrazole) MTT assay, a lactose dehydrogenase (LDH) assay, the use phosphorescent or fluorescent oxygen-sensitive probes, measuring total antioxidant activity or capacity, a glutathione reductase assay, and a Thiobarbituric Acid Reactive Substances (TBARS) assay. Certain embodiments provide methods of further testing those lead candidate agents that have been identified in the initial screening assay, in other model systems. The model systems include, but are not limited to, worms, flies, mammalian cells, fish, and warm-blooded animal models (e.g., a human α-synuclein transgenic mouse, recombinant adeno-associated virus exposed mammals, or rodents).

Accordingly, lead candidate agents identified in the in vitro screening assay may be further tested to identify those agents that promote α-synuclein disaggregation and/or prevent aggregation in non-mammalian organisms in vivo. Such organisms included but are not limited to C. elegans (worm), E. Coli (bacteria), Drosophila (fly), Danio rerio (zebrafish), Xenopus (frog), or Saccharomyces cerevisiae (yeast). An exemplary test includes using C. elegans neuronal cells which have been genetically modified to overexpress α-synuclein and whose dopaminergic neurons are fluorescent. Another exemplary test includes using transgenic C. elegans expressing wild-type or mutant human α-synuclein. Three point mutations have been identified far in human α-synuclein: A53T, A30P and E46K. In addition, duplication and triplication of the gene can result in α-synuclein aggregation and dysfunction. In C. elegans one manner of determining the effect of a candidate agent is to examine specific feeding behaviors which are affected in the absence of properly functioning dopaminergic neurons and which can be readily observed. The action of any agent can be revealed in real time, through a microscope. The C. elegans nervous system has neuronal cells similar to those found in humans, but in much smaller numbers. The process may be monitored from remote locations. Lead candidate agents identified from the C. elegans assay may then be tested in neurons from other non-mammalian model systems. In some embodiments, zebrafish (Danio rerio) can be used to probe the dopaminergic system and the effect of a candidate agent on neuronal function, and associated swimming behaviors and touch responses. In yet other embodiments, other model systems such as but not limited to Drosophila, Xenopus, or Saccharomyces cerevisiae can be utilized. Overexpressing can include genetically, non-genetically, transiently, permanently, by any method by which a cell or animal expresses greater α-synuclein than it normally does.

Furthermore, candidate agents identified in the non-mammalian in vivo model assay may be further tested to identify those agents that promote α-synuclein disaggregation and/or prevent aggregation in non-primate mammalian organisms in vivo. Transgenic mice that overexpress α-synuclein and display α-synuclein aggregation have been developed. See, e.g., Masliah et al., (2000) Science: 287 (1265-69). Using these genetically altered mice, experiments can be conducted quickly and efficiently to determine which agents may have desired effects in the neuronal cells of a warm-blooded vertebrate. In many instances, compounds tested in transgenic mice or other mammalian models will first be screened for potential α-synuclein aggregation modulating activity in a non-mammalian test system. For example, expression of human wild-type, A53T and/or A30P α-synuclein has been achieved in yeast, Drosophila, and C. elegans. Other examples can include expression of other point mutations such as E46K or expanded α-synuclein repeat regions, or extra copies of α-synuclein. Such examples are not limiting examples of non-mammalian systems which can be utilized in the practice of the disclosed invention. During this phase, the mice with excessive α-synuclein aggregation will be treated with the agents and then examined to identify those that prevent, inhibit or reverse aggregation.

Alternatively, other models which mimic damage to dopaminergic neurons may be utilized in addition to or in lieu of a non-mammalian organisms overexpressing α-synuclein, a mammalian organism overexpressing α-synuclein, a transgenic animal model, or a non-human primate overexpressing α-synuclein. Such a model could also be used in lieu of overexpressing α-synuclein in non-mammalian organisms. In some embodiments damage is caused by inducing a lesion or localized region of cell death, apoptosis or necrosis. Damage to dopaminergic neurons can take place in non-mammalian organisms, mammalian organisms, and non-human primates. In some embodiments, damage to neurons is achieved upon administering a neurotoxin. One such example is l-methyl-4phenyl-1,2,3,6-tetrahydropyridine (MPTP) treatment of neuronal cells, neuronal cell lines, and animals (Kowall et al, Clinical Neuroscience and Neuropathology, Vol 11, No. 1 Jan. 2000). MPTP is a potent Parkinsonian neurotoxin that causes selective degeneration of dopaminergic neurons in the substantia nigra. Indeed, MPTP-treated rodents are a standard Parkinson's disease model. See, e.g., Dauer and Przedborski, (2003) Neuron: 39 (889-909). Another example is 6-hydroxydopamine (6-OHDA) treatment of neuronal cells, neuronal cell lines, and animals (Cao et al., Neurobiology of Disease, (2005), 25(15): 3801-3812; McKinley et al., Molecular Brain Research (2005) 141: 128-137). There are several other neurotoxins well known to the person skilled in the art (Scober, Cell Tissue Res (2004) 318: 215-224). Neurotoxins can be introduced by a variety of methods including but not limited to: intracranial injection, orally, via drinking water or food, subcutaneously, intraperitoneally, intravenously, epidural injection, or intranasally. Such neurotoxin treatment has been shown to result in α-synuclein fibrillation, aggregation, and Parkinson's-like symptoms. In some embodiments, a neurotoxin is administered to a non-mammalian organism. In other embodiments a neurotoxin is administered to a mammalian organism. In other embodiments a neurotoxin is administered to a non-human primate or a primate.

Another model utilizes adeno-associated viruses (AAV). Recombinant AAV (e.g., AAV2 and AAV6 or any other serotype) expressing human α-synuclein (wild-type or mutant) can be targeted to the substantia nigra of test animals (e.g., rats, mice, non-human primates). See, e.g., Furler et al., (2001) Gene Ther.: 8 (864-73). Targeted overexpression of human α-synuclein using viral-vector mediated gene delivery into the substantia nigra of rats, mice and non-human primates leads to dopaminergic cell loss and the formation of α-synuclein aggregates reminiscent of Lewy bodies. Recombinant AAV expressing α-synuclein stereotaxically injected into the substantia nigra of mice, leads to a 25% reduction of dopaminergic neurons after 24 weeks of transduction. Furthermore, findings suggest that targeted overexpression of α-synuclein can induce pathology at the gross anatomical and molecular level in the substantia nigra, providing animal models in which upstream changes in Parkinson's disease pathogenesis can be further elucidated.

Accordingly, the present invention provides a method of using animal models for testing and screening for candidate agents that modulate a phenomenon associated with α-synuclein aggregation. The method comprises the steps of: (a) administering at least one or more candidate agents to a test animal generated by a method comprising (i) inducing α-synuclein aggregation (e.g., by overexpression of α-synuclein or by damaging dopaminergic neurons) in said test animal, (ii) allowing said test animal to develop α-synuclein aggregates, and (iii) administering candidate agents to the animal; and (b) determining the effect of said agents upon a phenomenon associated with α-synuclein aggregation. In some embodiments, the α-synuclein-related pathology and/or aggregation can be indirectly measured, by observing, measuring or quantifying cell physiology, neuronal function, and animal behavior. In some embodiments, axonal function, neuronal and glial transporter mechanisms, neuronal physiology and electrophysiology will be examined, utilizing methods known in the art. In other embodiments behaviors will be measured: examples of such behaviors include but are not limited to locomotor behavior, feeding behavior, swimming behavior, touch response or any other ethological animal behavior. Detection can also involve biochemical assays discussed herein. In certain embodiments the method involves performing at least two screening steps using a model system from lowest complexity to higher complexity.

The animal models of the present invention encompass any non-human vertebrates that are amenable to procedures yielding α-synuclein aggregates in the animal's nervous systems including the central and peripheral nervous system. Common model organisms include but are not limited to mammals, primates, and rodents. Non-limiting examples of the preferred models are rats, mice, guinea pigs, cats, dogs, rabbits, pigs, chimpanzees, and monkeys. The test animals can be wild-type or transgenic. Non human primates include but are not limited to gorilla, baboon, chimpanzee, macaque, rhesus monkey, squirrel monkey.

In one aspect, the subject method employs a transgenic animal having stably integrated into the genome a transgenic nucleotide sequence encoding wild-type or mutant α-synuclein encoding sequences. Examples of such transgenic animals are known and described. See, for example, St. Martin, et al., (2007) J. Neurochem.: 100 (1449-57); Mitchell, et al., (2007) Cell Transplant.: 16 (461-74); Wakamatsu, et al., J. Neurosci Res.: 85 (1819-25). In another aspect, the subject method involves a transgenic animal having an altered expression of at least one other gene, wherein upon expression of α-synuclein, the animal exhibits a greater degree of α-synuclein aggregation relative to a transgenic animal having a stably integrated transgenic nucleotide sequence encoding α-synuclein alone.

Expression of the transgene(s) carried in the transgenic animal may be inducible to effect expression that is ectopical, tissue specific, cell type specific, or even organelle specific. In some embodiments, tissue specific and cell specific regulatory sequences are available for expressing transgenes in the central nervous systems. Where expression of the transgene in particular subcellular location is desired, the transgene can be operably linked to the corresponding subcellular localization sequences by recombinant DNA techniques widely practiced in the art. Exemplary subcellular localization sequences include but are not limited to (a) a signal sequence that directs secretion of the gene product outside of the cell; (b) a membrane anchorage domain that allows attachment of the protein to the plasma membrane or other membranous compartment of the cell; (c) a nuclear localization sequence that mediates the translocation of the encoded protein to the nucleus; (d) an endoplasmic reticulum retention sequence (e.g. KDEL sequence) that confines the encoded protein primarily to the ER; or (e) any other sequences that play a role in differential subcellular distribution of a encoded protein product.

The present invention contemplates transgenic animals that carry one or more desired transgenes in all their cells, as well as animals which carry the transgenes in some, but not all their cells, i.e., mosaic animals. Animals of any species, including, but not limited to, mice, rats, rabbits, guinea pigs, pigs, micro-pigs, goats, and non-human primates, e.g., baboons, monkeys, and chimpanzees may be used to generate the subject transgenic animals.

A desired transgene may be integrated as a single copy or in concatamers, e.g., head-to-head tandems or head-to-tail tandems. The desired transgene may also be selectively introduced into and activated in a particular tissue or cell type, preferably cells within the central nervous system. The regulatory sequences required for such a cell-type specific activation will depend upon the particular cell type of interest, and will be apparent to those of skill in the art. Preferably, the targeted cell types are located in the nervous systems, including the central and peripheral nervous systems.

When it is desired that the transgene be integrated into the chromosomal site of the endogenous counterpart, gene targeting is preferred. Briefly, when such a technique is to be utilized, vectors containing some nucleotide sequences homologous to the endogenous counterpart are designed for the purpose of integrating, via homologous recombination with chromosomal sequences, into and disrupting the function of the nucleotide sequence of the endogenous gene.

Advances in technologies for embryo micromanipulation now permit introduction of heterologous DNA into fertilized mammalian ova as well. For instance, totipotent or pluripotent stem cells can be transformed by microinjection, calcium phosphate mediated precipitation, liposome fusion, retroviral infection or other means. The transformed cells are then introduced into the embryo, and the embryo will then develop into a transgenic animal. In a preferred embodiment, developing embryos are infected with a viral vector containing a desired transgene so that the transgenic animals expressing the transgene can be produced from the infected embryo. In another preferred embodiment, a desired transgene is co injected into the pronucleus or cytoplasm of the embryo, preferably at the single cell stage, and the embryo is allowed to develop into a mature transgenic animal. These and other variant methods for generating transgenic animals are well established in the art and hence are not detailed herein. See, for example, U.S. Pat. Nos. 5,175,385 and 5,175,384.

The transgenic animals of the present invention can be broadly categorized into two types: “knockouts” and “knockins”. A “knockout” has an alteration in the target gene via the introduction of transgenic sequences that results in a decrease of function of the target gene, preferably such that target gene expression is insignificant or undetectable. A “knockin” is a transgenic animal having an alteration in a host cell genome that results in an augmented expression of a target gene, e.g., by introduction of an additional copy of the target gene, or by operatively inserting a regulatory sequence that provides for enhanced expression of an endogenous copy of the target gene. The knock-in or knock-out transgenic animals can be heterozygous or homozygous with respect to the target genes. Both knockouts and knockins can be “bigenic”. Bigenic animals have at least two host cell genes being altered.

In one embodiment, the non-human transgenic animal comprises a transgenic nucleotide sequence comprising α-synuclein stably integrated into the genome. In some embodiments, the transgenic nucleotide sequence comprises a wild-type α-synuclein. In other embodiments, the transgenic nucleotide sequence comprises a mutant α-synuclein. In still other embodiments, a preferred transgenic animal exhibits an increased vulnerability to α-synuclein aggregation.

In one embodiment, subsequent to testing in non-primate animals, lead candidate agent(s) may be administered to a non-human primate to confirm the benefit of the compound. Such non-human primates may be genetically modified to overexpress α-synuclein (e.g., wild-type, A30P, A53T, or E46K) as in the non-primate animal. Alternatively, the non-human primates may be exposed to viral vectors (e.g., AAV) which lead to expression or overexpression of human α-synuclein. See, e.g., Yasuda et al., (2007) Neurosci.: 144 (743-53). Additionally or alternatively, non-human primates may be treated with MPTP, 6-OHDA or any other neurotoxin to induce Parkinson's disease-like state prior to testing the candidate agents. See, e.g., Redmond et al., (2007) Proc. Nat'l Acad. Sci.: 104 (12175-80). Once a lead candidate agent has proved effective in non-human primates, small scale human clinical trials may be commenced.

As previously noted, α-synuclein useful in the methods and systems of the invention includes mutant and wild-type polypeptides, and fragments thereof provided that such fragments are capable of participating in the formation of aggregates. Fragments of α-synuclein include, for example, the non-amyloid component (NAC) fragment that is a constituent of Alzheimer's disease amyloid plaques. Any of these forms of α-synuclein may be produced for purification by transformation of appropriate organisms (e.g., bacteria, yeast, Drosophila, etc.) using standard molecular biological techniques.

Exemplary mutants of α-synuclein include two different point mutations in the α-synuclein gene (A53T and A30P) that were identified in separate families with dominantly transmitted Parkinson's disease (see, e.g., international application WO 98/5950, published Dec. 30, 1998). These point mutations of α-synuclein have been shown to increase the ability of a synuclein to aggregate and to slow down degradation of the mutated a synuclein. The consistent effect of these mutations in increasing the amount and aggregation of α-synuclein suggests that these processes play an important role in the pathophysiology of various neurodegenerative disorders. Other mutant forms of human α-synuclein have been described (e.g. E46K). One of skill in the art will recognize that any of the transgenic organisms or cells discussed above can comprise any of these forms of human α-synuclein (e.g., wild-type, A30P, A53T, or E46K) or any other mutant, truncated, chemically modified, cDNA, or other relevant form of the human α-synuclein gene. Furthermore, standard procedures can be utilized to produce and purify any of these forms of human α-synuclein for use in cell-free assays. See, e.g., Jakes et al., (1994) FEBS Lett.: 345 (27-32). While these mutant forms of α-synuclein are associated with aggregation, overexpression of wild-type α-synuclein is associated with cellular toxicity in multiple organisms, including yeast, Drosophila, and mice. Thus, agents that inhibit, reverse, or otherwise modulate wild-type or mutant human α-synuclein aggregation, represent a novel therapeutic strategy as disease-modifying agents for neurodegeneration.

Referring again to FIGS. 1 and 2, the invention provides systems for identifying an agent that modulates α-synuclein aggregation. Such a system may include mechanisms for performing various parts of a method for identifying an agent that that modulates α-synuclein aggregation at remote locations. For example, candidate agents may be screened in an in vitro assay for activity associated with promoting α-synuclein disaggregation, or inhibiting α-synuclein aggregation. The in vitro screen may be performed at a first location and the results transmitted to a second or central location. The results may be accessed by multiple users such that subsequent in vivo assays may be performed using those agents identified by the in vitro assay as possessing desirable activities against α-synuclein aggregation. The results of the in vivo assays may similarly be transmitted to a central location for storage and access by other users. Accordingly, the information generated by multiple users at various locations can be communicated to a central location, such as a server, and compiled. The compilation of assay information can entail the construction of databases that reflect the results of the various in vitro and in vivo assays performed on the plurality of candidate agents. Thus, the invention further provides a computer system including a database incorporating records of the activity of any or all of a plurality of agents that may modulate α-synuclein aggregation. Such a database including results inputted by one or more users of the database; a processor for cross-assembling the results; a processor for correlating the information and for generating an index of agents that promote α-synuclein disaggregation or inhibit α-synuclein aggregation, and a means for outputting to an output device the results of the index.

It is therefore immediately evident that a computer program designed for storing, correlating and indexing the results of the in vitro and in vivo assays may be provided which is suitable for use with the methods and systems of the present invention. Accordingly, the invention provides methods and systems for indexing the results of various screening assays and identifying those agents that may be used in a therapeutic composition for treating a disorder or condition associated with α-synuclein aggregation.

It is also envisioned that the method and systems of the present invention can be provided in a kit form for use for screening and candidate agent selection, regardless of the source of the candidate agents to be screened.

It is further envisioned that the method and systems of the present invention can be integrated with other methods of identifying agents suitable for modulating α-synuclein aggregation. For example, a wide variety of structural, chemical, and sequence information is available for agents that may be screened by a method or system of the invention. For example, structural information for polypeptides can be generated in-silico based solely on the amino acid content of the polypeptide. Similarly, crystallographic structures of candidate polypeptides are available through various databases known to the skilled artisan. Chemical information for small molecules that may be screened by the present methods and systems are also available from various databases. In addition, sequence information for nucleic acid molecules, such as antisense RNA or siRNA (small interfering RNA), is similarly available through various databases. The integration of the information generated by a method or system of the present invention can be correlated with the information contained in such databases in order to design agents that not only modulate α-synuclein aggregation, but also are suitable for administration to a patient. Thus, cross-indexing the information generated in each phase of a method or system provided herein with information contained in other databases facilitates that identification of agents suitable for use as a therapeutic. For example, the information generated by the invention can be compared to other indices in order to facilitate the identification of common structures, features or sequences of agents that modulate α-synuclein aggregation.

Accordingly, aspects of the invention may be implemented in hardware or software, or a combination of both. The algorithms and processes of the invention may be implemented in one or more computer programs executing on programmable computers each comprising at least one processor, at least one data storage system (including volatile and non-volatile memory and/or storage elements), at least one input device, and at least one output device. Program code is applied to input data to perform the functions described herein and generate output information. The output information is applied to one or more output devices, in known fashion.

Each program may be implemented in any desired computer language (including machine, assembly, high level procedural, or object oriented programming languages) to communicate with a computer system. In any case, the language may be a compiled or interpreted language.

Each such computer program is preferably stored on a storage media or device (e.g., ROM, CD.-ROM, tape, or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

As previously noted, the invention provides methods and systems for identifying those agents that may be used in a therapeutic composition for treating a disorder associated with α-synuclein aggregation. In certain embodiments, candidate agents (i.e., drugs or compounds) may be formulated in combination with a suitable pharmaceutical carrier. Such formulations comprise a therapeutically effective amount of the agent, and a pharmaceutically acceptable carrier (excipient). Examples of suitable carriers are well known in the art. To illustrate, the pharmaceutically acceptable carrier can be an aqueous solution or physiologically acceptable buffer. Optionally, the aqueous solution is an acid buffered solution. Such acid buffered solution may comprise hydrochloric, sulfuric, tartaric, phosphoric, ascorbic, citric, fumaric, maleic, or acetic acid. Alternatively, such carriers include, but are not limited to, saline, buffered saline, dextrose, water, glycerol, ethanol, and combinations thereof. Formulations will suit the mode of administration, and are well within the skill of the art.

In certain embodiments of such methods, one or more agents can be administered, together (simultaneously) or at different times (sequentially). In addition, such agents can be administered with another type(s) of drug(s) for treating a disease associated with α-synuclein aggregation. For example, the identified agent may be administered together with Levodopa (L-DOPA) for treating Parkinson's disease.

The phrase “therapeutically effective amount,” as used herein, refers to an amount that is sufficient or effective to prevent or treat (delay or prevent the onset of, prevent the progression of, inhibit, decrease or reverse) a disease or condition associated with α-synuclein aggregation, including alleviating symptoms of such diseases.

The dosage range depends on the choice of the agent, the route of administration, the nature of the formulation, the nature of the subject's condition, and the judgment of the attending practitioner. Wide variations in the needed dosage, however, are to be expected in view of the variety of drugs available and the differing efficiencies of various routes of administration. For example, oral administration would be expected to require higher dosages than administration by intravenous injection. Variations in these dosage levels can be adjusted using standard empirical routines for optimization, as is well understood in the art.

Various delivery systems are known and can be used to administer a biologically active agent of the invention, e.g., encapsulation in liposomes, microparticles, microcapsules, expression by recombinant cells, receptor-mediated endocytosis (see, e.g., Wu and Wu, (1987), J. Biol. Chem. 262:4429-4432), construction of a therapeutic nucleic acid as part of a retroviral or other vector, etc. Methods of delivery include but are not limited to intra-arterial, intra-muscular, intravenous, intranasal, intracerebral and oral routes. In a specific embodiment, it may be desirable to administer the pharmaceutical compositions of the invention locally to the area in need of treatment; this may be achieved by, for example, and not by way of limitation, local infusion during surgery, by injection, by means of a catheter, or implantation of a device coated with the agent and a slow-release mechanism. In certain embodiments, the agents are delivered to a subject's nervous systems, preferably the central nervous system. In another embodiment, the agents are administered to neuronal tissues undergoing, or suspected of undergoing α-synuclein deposition.

Administration of the selected agent can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician.

The preparation of pharmaceutical compositions of this invention is conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it may be desirable to process the active ingredient further in the preparation of pharmaceutical compositions. Appropriate processing may include mixing with appropriate non-toxic and non-interfering components, sterilizing, dividing into dose units, and enclosing in a delivery device.

Pharmaceutical compositions for oral, intracerebral, intramuscular, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or aerosol when used with an appropriate nebulizer device.

Pharmaceutically acceptable liquid compositions can, for example, be prepared by dissolving or dispersing an agent embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

Where desired, the pharmaceutical compositions can be formulated in slow release or sustained release forms, whereby a relatively consistent level of the active compound are provided over an extended period.

### EXAMPLES

### Example 1

### In Vitro Cell Free Analysis of Compounds for α-Synuclein Anti-Aggregation Activity

In this example, one procedure to test for the ability of candidate agents to inhibit or reverse α-synuclein aggregation is described. Recombinant human α-synuclein (either wild-type or mutant—e.g. A53T, A30P, or E46K) can be expressed and purified as previously described (see, e.g., Jakes et al., (1994) FEBS Lett.: 345 (27-32)). Purified α-synuclein samples will be concentrated and centrifuged for 10 minutes at 100,000×g to ensure removal of aggregates formed during concentration. In assays involving analysis of a test compound to reverse aggregation, these removed aggregates can be utilized. Following removal of the aggregates, samples can be suspended to an appropriate final concentration (e.g., 7 mg/ml) in Tris-buffered saline (20 mM Tris, pH 7.5, and 0.2 M NaCl). An appropriate volume of the purified recombinant human α-synuclein suspension is combined with an appropriate volume of a test compound. Volumes may need to be adjusted for individual agents and test samples due to the chemical and/or physical characteristics (e.g., solubility, molecular weight, etc.) of a given candidate agent. Such variability is readily appreciated by one skilled in the art and multiple approaches to maintaining final test sample concentration are well known in the art.

Samples containing both recombinant human α-synuclein and the candidate agents are incubated at 37° C. As a negative control, a sample containing only recombinant human α-synuclein is incubated under identical conditions. As a positive control, an additive containing a known inhibitor of recombinant human α-synuclein aggregation is added and incubated under identical conditions. Aliquots are extracted at various time points and the samples are examined by appropriate methods (e.g. ThioflavinT fluorescence, scanning electron microscopy, Western blot, fluorescent anti-α-synuclein antibody staining, etc) to determine the extent, if any, of anti-aggregation activity. An arbitrary, but selective, value will be applied to determine whether an individual candidate agent is “positive” for anti-aggregation activity. A similar approach can be used to determine the ability of a candidate agent to reverse aggregation of pre-aggregated recombinant human α-synuclein. Those agents which are determined to be “positive” for anti-aggregation (or disaggregation) activity will be further examined for in vivo activity. Although this example involves the use of a cell-free system, one of skill in the art will recognize that cell-containing in vitro system can also be utilized. Such cell containing in vitro systems could be proliferating cell lines, immortalized cell lines, or cells taken from an organism.

### Example 2

### Analysis of Compounds for α-Synuclein Anti-Aggregation Activity in Neuronal Cells

In this example, a procedure to test for the ability of candidate agents to inhibit or reverse α-synuclein aggregation in neuronal or neuronal-like cells is described. Incorporation of this procedure in the screening approach would help further identify lead compounds prior to commencing testing in model organisms or non-human primates. Primary neuronal cultures containing dopaminergic cells would be prepared. The SH-SY5Y cell lines can be utilized (American Type Culture Collection (ATCC) or other resources). Following culturing under optimal conditions, wild-type or mutant α-synuclein will be overexpressed in the cells. Following an appropriate time interval, fibrillation and/or aggregation will be observed. Candidate agent or appropriate vehicle will be applied to before, during, of at specified times following over expression of α-synuclein. Mutant or wild-type α-synuclein can be delivered by transfection methods, or by viral vectors to the neuronal cells. Modified herpes viral vectors (HSV1 or HSV2) can be employed to overexpress wild-type of mutant α-synuclein, as modified HSV preferentially infects neuronal cells, thus better mimicking the selective vulnerability of neuronal cells to α-synuclein mediated aggregation.

### Example 3

### Analysis of Anti-Aggregation Activity in Human α-Synuclein Expressing Transgenic C. elegans

To test the in vivo efficacy of candidate agents that are determined to have anti-aggregation activity in vitro, a first model using transgenic C. elegans (roundworm) overexpressing human α-synuclein will be utilized. Such transgenic worms have been described in the literature. See, e.g., Lakso et al., (2003) J. Neurochem.: 86 (165-72); Kuwarara et al., (2006) J. Biol. Chem.: 281 (334-40). Briefly, such worms may be generated by cloning the dat-1 (a dopamine transporter) promoter, a 719-bp region containing the initiation codon, from C. elegans genomic DNA by polymerase chain reaction (PCR). The PCR product is inserted into a BamHI/NotI site of the enhanced green fluorescence protein (EGFP) vector pFXneEGFP. Full length cDNA encoding α-synuclein (wild-type, A53T or A30P) is digested with NotI/BglII and the resulting fragment is ligated into pFXneEGFP containing the dat-1 sequence. To generate transgenic C. elegans, dat-1::α-synuclein plasmid DNA is mixed with ges-1::red fluorescent protein plasmid DNA (for determination of uptake of transgenic DNA) and injected into the gonads of young adult hermaphrodite N2 Bristol (wild-type) worms. Transgenes are integrated into chromosomes by UV irradiation and isolated positive worms are outcrossed 2-4 times.

Expression of human α-synuclein in such transgenic worms has been shown to be localized to dopamine neurons by immunohistochemistry. See, e.g., Kuwarara et al., (2006) J. Biol. Chem.: 281 (334-40). Additionally, these transgenic worms exhibit clumping bodies which are positively immunoreactive for human α-synuclein. Furthermore, such transgenic worms show an increased tendency to phosphorylate α-synuclein at Ser-129, a form characteristic of α-synuclein in Lewy bodies in humans and transgenic mice. See, e.g., Fujiwara et al., (2002) Nat. Cell Biol.: 4 (160-64); Kahle et al., (2002) EMBO Rep.: 3 (583-88).

C. elegans moves by bending its body and the frequency of the bending motion determines the speed with which the worm moves. When wild-type worms detect food (e.g., bacteria), the frequency of bending (locomotion) decreases as the worm slows down to feed. It has been shown that C. elegans strains which lack the ability to synthesize dopamine in dopamine neurons show specific failure in food-sensing behavior that is readily observable under the microscope (i.e., such worms do not decrease the bending motion). See, e.g., Sawin, et al., (2000) Neuron: 26 (619-31). Furthermore, this effect was also shown to occur in transgenic C. elegans expressing A53T, E46K, or A30P human α-synuclein. See, e.g., Kuwarara et al., (2006) J. Biol. Chem.: 281 (334-40). Exogenous dopamine reversed these effects, suggesting that restoration of dopamine signaling reverses the damaging effects of human α-synuclein.

In this example, one approach to determining the anti-aggregation capabilities of candidate agents which showed activity in vitro is to expose transgenic C. elegans expressing A53T, E46K, or A30P human α-synuclein to the test compounds of interest. Microscopic evaluation of feeding pattern behavior can be used to determine whether an agent can reverse the effects of the transgene expression.

As mentioned above, transgenic C. elegans expressing wild-type, A53T, E46K, or A30P human α-synuclein show the presence of clumping bodies which are immunoreactive with human α-synuclein. Thus, in a second approach in this example, the transgenic worms could be exposed to the candidate agents to determine their effects, if any, on the clumping bodies. The animals would be sacrificed and sections can be prepared and stained with appropriate immunohistochemical protocols. Candidate agents which resulted in lessening of the α-synuclein clumping bodies (either in number or size or frequency) could be considered a possible candidate for further testing in a mammalian model.

### Example 4

### Analysis of Anti-Aggregation Activity in Human α-Synuclein Overexpressing Transgenic Mice

To test the efficacy of the candidate agents which showed positive results in a transgenic C. elegans model and/or in an in vitro model, transgenic mice overexpressing human α-synuclein can be utilized. Such mice have been constructed and described. See, e.g., Masliah et al., (2000) Science: 287 (1265-69); Masliah et al., (2001) Proc. Nat'l Acad. Sci.: 98 (12245-50). Briefly, such mice can be constructed by placing a fragment of 5′-flanking region of the human PDGF-β chain gene (which targets expression of human proteins to neurons) upstream of a NotI/SalI DNA fragment containing an SV40 splice, the full length cDNA encoding wild-type (or mutant) human α-synuclein, and SV40 polyadenylation signal. This construct can be microinjected into one-cell embryos of C57BL/6 mice according to protocols well known in the art. The embryos are implanted and potential transgenic mice are screened by standard protocols.

Transgenic mice overexpressing human α-synuclein exhibit prominent intraneuronal inclusions which are immunoreactive with human α-synuclein, but not mouse α-synuclein. These inclusions grow as the mice age. These inclusions resemble Lewy bodies in appearance, structure and anatomical position within the brain (i.e. in deep layers of the neocortex and in dopaminergic neurons of the substantia nigra). Furthermore, in mice expressing the highest levels of human α-synuclein, a marked reduction in tyrosine hydroxylase (TH) positive neurons is seen. See, e.g., Masliah et al., (2000) Science: 287 (1265-69). TH is required for dopamine synthesis. Mice expressing high levels of human α-synuclein also perform poorly on the rotorod test (a motor performance test) compared to non-transgenic mice and mice expressing low levels of human α-synuclein. Thus, the loss of dopaminergic input to the striatum in the overexpressing transgenic mice correlates with Parkinson's disease related neurological impairments and motor function.

In this example, transgenic mice overexpressing human α-synuclein provide a good model for testing the ability to reduce human α-synuclein in a mammalian model of candidate agents which showed efficacy in C. elegans. The candidate agents are supplied to the overexpressing transgenic mice through a route appropriate for the individual agent. Such a route could be determined by one of skill in the art, but route of administration for an individual agent may need to vary based on the chemical nature of the candidate agent.

Multiple approaches can be taken to examine the efficacy of a candidate agent on the mice. Non-exclusive testing methods include: 1) examination of motor function using standard testing procedures can be used (e.g., the rotorod test); 2) animals can be sacrificed to perform immunohistochemical analysis of brain tissue for the effect of the agent, if any, on the intraneuronal inclusions or other histological features, and 3) other aspects of Parkinson's disease-related impairments, such as loss of function of dopaminergic neuron function and actual levels of dopamine produced can be examined. In some embodiments, cognitive abnormalities and olfactory dysfunction will also be tested. Additionally, a combination of these and other approaches can be used to determine which of the candidate agents are best suited for non-human primate trials, and eventually human clinical trials.

### Example 5

### Analysis of Anti-Aggregation Activity in Human α-Synuclein Overexpressing Rats Exposed to Recombinant Adeno-Associated Virus

To test the efficacy of candidate agents which showed positive results in a transgenic C. elegans model and/or in an in vitro model, rats overexpressing human α-synuclein encoded by recombinant AAV (rAAV) can be utilized. Such viruses and rats have been described. See, e.g., Furler et al., (2001) Gene Ther.: 8 (864-73). Briefly, comparably infectious titers of rAAV expressing human α-synuclein (wild-type, A53T, A30P, or E46K) and GFP linked by an IRES sequence and under control of the PDGF-f (which targets expression of proteins to neurons) are unilaterally injected into the substantia nigra of adult rats. Expression of α-synuclein is confirmed by detecting expression of GFP and/or other methods available in the art.

Rats positive for α-synuclein expression will be monitored for decreases in dopamine by direct measurement of dopamine levels in the substantia nigra (or other anatomical regions of interest). Human α-synuclein overexpressing rats will be exposed to appropriate amounts of candidate agents which showed positive results in the transgenic C. elegans model and/or in an in vitro model. The agents are supplied to the α-synuclein overexpressing rats through a route appropriate for the individual agent. Such a route can be determined by one of skill in the art, but route of administration for an individual candidate agent may need to vary based on the chemical and/or physical nature of the agent. Dopamine levels of rats expressing α-synuclein with and without treatment with candidate agents will be examined to determine the potential effectiveness of the agents in treating Parkinson's disease. Additionally, animals may be sacrificed to perform appropriate immunostaining of histological samples to detect changes in cellular morphology, gene expression, protein production, etc. Those agents which show promise in this mammalian model will be considered for further testing in non-human primates, and eventually, clinical trials in humans.

### Example 6

### Analysis of Anti-Aggregation Activity in Human α-Synuclein Overexpressing Primates Exposed to Recombinant Adeno-Associated Virus

To test the efficacy of candidate agents which showed positive results in the transgenic C. elegans model and/or the transgenic mouse and/or rAAV-exposed rats, macaques overexpressing human α-synuclein encoded by recombinant AAV (rAAV) can be utilized. Such viruses and primates have been described. See, e.g., Yasuda et al., (2007) Neurosci.: 144 (743-53). Briefly, comparably infectious titers of rAAV serotype-2 expressing human α-synuclein (wild-type, A53T, A30P, or E46K) and GFP linked by an IRES sequence and under control of the PDGF-β (which targets expression of proteins to neurons) are unilaterally injected into the striatum of macaque monkeys, resulting in expression of human α-synuclein in striatonigral GABAergic and nigrostriatal dopaminergic neurons. Expression of α-synuclein is confirmed by detecting expression of GFP and/or other methods available in the art. Expression of human α-synuclein has been shown to lead to accumulation of human α-synuclein and/or phosphorylation at the Ser129 residue.

Non-human primates (e.g. macaques or squirrel monkeys) positive for α-synuclein expression will be monitored for decreases in dopamine by direct measurement of dopamine levels in the substantia nigra (or other anatomical regions of interest). Human α-synuclein overexpressing macaques will be exposed to appropriate amounts of candidate agents which showed positive results in the transgenic non-mammalian organism and/or the transgenic and/or rAAV-exposed rodent models. The agents are supplied to the α-synuclein overexpressing non-human primates through a route appropriate for the individual agent. Such a route can be determined by one of skill in the art, but route of administration for an individual candidate agent may need to vary based on the chemical and/or physical nature of the agent. Dopamine levels of macaques expressing α-synuclein with and without treatment with candidate agents will be examined to determine the potential effectiveness of the agents in treating Parkinson's disease. Other endpoints relevant to Parkinson's Disease such as motoric behavior, mood and response to stimuli will be examined. Additionally, animals may be sacrificed to perform appropriate immunostaining of histological samples to detect changes in cellular morphology, gene expression, protein production, etc. Those agents which show promise in this primate model will be considered for further testing, and eventually, clinical trials in humans.

The examples set forth above are given to provide those of ordinary skill in the art with a complete disclosure and description of how to make and use various embodiments of the methods and systems disclosed herein, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

A number of embodiments of the invention have been described. Nevertheless, it will be understood that various modifications may be made without departing from the spirit and scope of the invention. Accordingly, other embodiments are within the scope of the following claims.

1-26. (canceled)

27. A method for identifying an agent, the method comprising:
a) providing a plurality of candidate agents;
b) contacting a plurality of candidate agents with a polypeptide comprising α-synuclein and identifying agents that promote α-synuclein disaggregation, or inhibit α-synuclein aggregation upon contact with said agent, in vitro;
c) administering one or more agents identified in b) to a non-mammalian organism exhibiting damaged dopaminergic neuronal cells which comprise α-synuclein and identifying agents that promote α-synuclein disaggregation, or inhibit α-synuclein aggregation; and
d) administering at least one agent identified in c) to a mammalian organism comprising neuronal cells that overexpress a polypeptide comprising α-synuclein and determining whether the agent promotes α-synuclein disaggregation, or inhibits α-synuclein aggregation.

28. A method for identifying an agent, the method comprising:
a) providing a plurality of candidate agents;
b) contacting a plurality of candidate agents with a polypeptide comprising α-synuclein and identifying agents that promote α-synuclein disaggregation, or inhibit α-synuclein aggregation upon contact with said agent, in vitro;
c) administering one or more agents identified in b) to a non-mammalian organism comprising neuronal cells that overexpress a polypeptide comprising α-synuclein and identifying agents that promote α-synuclein disaggregation, or inhibit α-synuclein aggregation; and
d) administering at least one agent identified in c) to a mammalian organism exhibiting damaged dopaminergic neuronal cells which comprise α-synuclein and determining whether the agent promotes α-synuclein disaggregation, or inhibits α-synuclein aggregation.

29. A method for identifying an agent, the method comprising:
a) providing a plurality of candidate agents;
b) contacting a plurality of candidate agents with a polypeptide comprising α-synuclein and identifying agents that promote α-synuclein disaggregation, or inhibit α-synuclein aggregation upon contact with said agent, in vitro;
c) administering one or more agents identified in b) to a non-mammalian organism exhibiting damaged dopaminergic neuronal cells which comprise α-synuclein and identifying agents that promote α-synuclein disaggregation, or inhibit α-synuclein aggregation; and
d) administering at least one agent identified in c) to a mammalian organism exhibiting damaged dopaminergic neuronal cells which comprise α-synuclein and determining whether the agent promotes α-synuclein disaggregation, or inhibits α-synuclein aggregation.

30. The method of claim 28 further comprising administering an agent identified in d) to a non-human primate overexpressing a polypeptide comprising α-synuclein and determining whether the agent promotes α-synuclein disaggregation, or inhibits α-synuclein aggregation.

31. The method of claim 28 further comprising administering an agent identified in d) to a non-human primate exhibiting damaged dopaminergic neuronal cells which comprise α-synuclein and determining whether the agent promotes α-synuclein disaggregation, or inhibits α-synuclein aggregation.

32. A method for identifying an agent, the method comprising:
a) providing a plurality of candidate agents;
b) contacting a plurality of candidate agents with a polypeptide comprising α-synuclein and identifying agents that promote α-synuclein disaggregation, or inhibit α-synuclein aggregation upon contact with said agent, in vitro; and
c) administering one or more agents identified in b) to a non-mammalian organism exhibiting damaged dopaminergic neuronal cells which comprise α-synuclein and determining whether the agent promotes α-synuclein disaggregation, or inhibits α-synuclein aggregation.

33. A method for identifying an agent, the method comprising:
a) providing a plurality of candidate agents;
b) contacting a plurality of candidate agents with a polypeptide comprising α-synuclein and identifying agents that promote α-synuclein disaggregation, or inhibit α-synuclein aggregation upon contact with said agent, in vitro; and
c) administering at least one agent identified in b) to a mammalian organism exhibiting damaged dopaminergic neuronal cells which comprise α-synuclein and determining whether the agent promotes α-synuclein disaggregation, or inhibits α-synuclein aggregation.

34. A method for identifying an agent, the method comprising:
a) providing a plurality of candidate agents;
b) administering a plurality of agents to a non-mammalian organism exhibiting damaged dopaminergic neuronal cells which comprise α-synuclein and identifying agents that promote α-synuclein disaggregation, or inhibit α-synuclein aggregation; and
c) administering at least one agent identified in b) to a mammalian organism comprising neuronal cells that overexpress a polypeptide comprising α-synuclein and determining whether the agent promotes α-synuclein disaggregation, or inhibits α-synuclein aggregation.

35. A method for identifying an agent, the method comprising:
a) providing a plurality of candidate agents;
b) administering a plurality of agents to a non-mammalian organism comprising neuronal cells that overexpress a polypeptide comprising α-synuclein and identifying agents that promote α-synuclein disaggregation, or inhibit α-synuclein aggregation; and
c) administering at least one agent identified in b) to a mammalian organism exhibiting damaged dopaminergic neuronal cells which comprise α-synuclein and determining whether the agent promotes α-synuclein disaggregation, or inhibits α-synuclein aggregation.

36. A method for identifying an agent, the method comprising:
a) providing a plurality of candidate agents;
b) administering a plurality of agents to a non-mammalian organism exhibiting damaged dopaminergic neuronal cells which comprise α-synuclein and identifying agents that promote α-synuclein disaggregation, or inhibit α-synuclein aggregation; and
c) administering at least one agent identified in b) to a mammalian organism exhibiting damaged dopaminergic neuronal cells which comprise α-synuclein and determining whether the agent promotes α-synuclein disaggregation, or inhibits α-synuclein aggregation.

37. The method of claim 33 further comprising administering an agent identified in c) to a non-human primate overexpressing a polypeptide comprising α-synuclein and determining whether the agent promotes α-synuclein disaggregation, or inhibits α-synuclein aggregation.

38. The method of claim 33 further comprising administering an agent identified in c) to a non-human primate exhibiting damaged dopaminergic neuronal cells which comprise α-synuclein and determining whether the agent promotes α-synuclein disaggregation, or inhibits α-synuclein aggregation.

39. The method of claim 33 further comprising communicating the identified compounds to a database.

40. The method of claim 33 wherein the promotion of disaggregation, or the inhibition of aggregation, by the agent identifies the agent as a compound suitable for treating a condition associated with α-synuclein aggregation.

41. The method of claim 33 wherein the agent is selected from the group consisting of a chemical, a small molecule, a biomolecule, and a virus.

42. A method of treating an individual suffering from a disease associated with α-synuclein aggregation, the method comprising administering to the individual a pharmaceutical composition comprising a therapeutically effective amount of an agent identified by the method of claims 27 to 38.

43. The method of claim 42 wherein the disease is Parkinson's Disease.

44. The method of claim 37 wherein the overexpression of α-synuclein is achieved by AAV-mediated delivery of α-synuclein.

45. The method of claim 44 wherein the α-synuclein is mutant α-synuclein and is selected from the group consisting of A53T, E46K, and A30P.

46. The method of claim 33 or 38 wherein the damage to dopaminergic neurons is result of neurotoxin administration.

47. The method of claim 46 wherein the neurotoxin is MPTP or 6-OHDA.

48. The method of claim 33 wherein the mammalian organism is a rodent.

49. The method of claim 33 wherein the mammalian organism is transgenic.

50. The method of claim 49 wherein the transgenic mammalian organism is a rat.

51. The method of claim 28 wherein the non-mammalian organism is selected from the group consisting of worm, fly, fish, yeast, and bacteria.

52. The method of claim 28 wherein the non-mammalian organism is a worm and the worm is C. elegans.

53. The method of claim 28 wherein the non-mammalian organism is a fly and the fly is D. melanogaster.

54. The method of method of claim 28 wherein the non-mammalian organism is a fish and the fish is D. rerio.

55. The method of claim 33 wherein the determining of whether the agent promotes α-synuclein disaggregation, or inhibits α-synuclein aggregation is by microscopy.

56. The method of claims 28 or 33 wherein the determining of whether the agent promotes α-synuclein disaggregation, or inhibits α-synuclein aggregation comprises detecting a change in behavior.

57. The method of claim 56 wherein the behavior is selected from the group consisting of locomotor behavior, swimming behavior, touch response, and feeding behavior.

58. The method of claim 33 wherein the determining of whether the agent promotes α-synuclein disaggregation, or inhibits α-synuclein aggregation comprises measuring a change in oxidative stress or mitochondrial function.

59. The method of claim 33 wherein the determining of whether the agent promotes α-synuclein disaggregation, or inhibits α-synuclein aggregation comprises measuring a change neuronal physiology.

60. The method of claim 59 wherein the neuronal physiology measured is selected from the group consisting of: axonal function, transporter mechanisms, and electrophysiology.

61. The method of claim 37 or 38 wherein the non-human primate is selected from the group consisting of: gorilla, baboon, chimpanzee, macaque, rhesus monkey, and squirrel monkey.

62. The method of claim 33 wherein the in vitro system is a primary neuronal culture or a neuronal cell line.

63. (canceled)

64. A kit for the screening of a library of candidate agents comprising:
a) purified α-synuclein; and
b) components of a cell-free system

65. A kit for the screening of a library of candidate agents comprising:
a) purified α-synuclein or a plasmid for overexpression of α-synuclein; and
b) a library of candidate agents
